(19) United States
(12) Patent Application Publication
(10) Pub. No.: US 2015/0039060 A1
(43) Pub. Date: Feb. 5, 2015

(54) WEARABLE AND BREATHABLE PHOTO THERAPY PATCH

(71) Applicants: Elvira Johanna Maria Paulussen, Reppel-Bocholt (BE); Guofu Zhou, Best (NL); Marjolein Yvonne Jansen, Eindhoven (NL); Tim Dekker, Eindhoven (NL); Lucas Johannes Anna Maria Beckers, Veldhoven (NL); Liesbeth Van Pieterson, Heeze (NL)

(72) Inventors: Elvira Johanna Maria Paulussen, Reppel-Bocholt (BE); Guofu Zhou, Best (NL); Marjolein Yvonne Jansen, Eindhoven (NL); Tim Dekker, Eindhoven (NL); Lucas Johannes Anna Maria Beckers, Veldhoven (NL); Liesbeth Van Pieterson, Heeze (NL)

(73) Assignee: KONINKLIJKE PHILIPS ELECTRONICS N.V., EINDHOVEN (NL)

(21) Appl. No.: 13/995,186

(22) PCT Filed: Dec. 16, 2011
(86) PCT No.: PCT/IB11/55748

§ 371 Date: Nov. 19, 2013

(30) Foreign Application Priority Data
Dec. 21, 2010 (EP) 10196115.9

(51) Int. Cl. A61N 5/06

(52) U.S. Cl. CPC A61N 5/0616; A61N 2005/0666; A61N 2005/0643
USPC 607/90

(57) ABSTRACT

A radiation device (30) for providing radiation to the skin is described. The radiation device comprises a radiation guide (31) for directing radiation whereby the radiation guide (31) is configured for receiving radiation from at least one radiation source (20) in a side-lit configuration. The radiation device (30) comprises moisture transfer channels (32) inside the radiation guide (31) for enabling moisture transfer from the skin through the radiation guide (31) to the environment. The shape of the walls of the moisture transfer channels (32) is adapted for redirecting radiation in the radiation guide (31) towards the skin.

### FIELD OF THE INVENTION

The present invention relates to radiation devices. More particularly, the present invention relates to wearable devices for efficiently and comfortably providing radiation therapy towards the skin.

### BACKGROUND OF THE INVENTION

Radiation therapy devices use radiation to induce effects on the skin. Such effects may for example be cosmetic, overall wellness or medical, such as for pain relief. Examples of applications are treatment of skin rejuvenation, acne control, psoriasis, use of infrared products, massage devices or wellness lamps, and use of heat for pain relief. Nowadays heat therapy is a well established and numerous products such as heat cabins and flood lamps are on the market. Most devices are based on infrared (IR) light. The benefits of infrared as heat therapy are based on a vasodilatory response in the skin which locally enhances the blood circulation. This results in an increased metabolic rate and transport of metabolites and other essential biochemical compounds. Benefits are also gained by deeper penetration of heat, providing a gentle and pleasant warming effect.

Whereas conventional systems such as incandescent lamps with an infrared filter still are widely applied, wearable radiation therapy products are also largely investigated. The use of LEDs as radiation source offers a compact solution for wearable radiation devices, and has many other advantages, such as the fact that LEDs can be switched on and off very fast and in a spatial sequence, giving a massage sensation not only in time but also in space, that different, well-defined wavelength and power ranges available in the LED spectrum can be used for well defined treatments on the skin (i.e. a combination of two or more wavelength ranges and or power ranges), and that LEDs are point sources allowing redirection of the radiation produced in the device. In order to guide the radiation from a compact radiation source to the skin, typically two radiation configurations can be used. In direct-lit radiation, the radiation sources may typically be embedded throughout the patch and can directly irradiate the skin, whereas in side-lit configuration, the radiation sources are positioned at sides of the patch, and their radiation is transported lateral across the device before out-coupling to the skin.

In both configurations, the radiation devices require a spacer between the LEDs and the skin, which may be an intermediate optical layer. A material that is often used in healthcare applications in contact with the skin, is silicone. The safety of silicone as interaction layer with the skin has been proven thoroughly. Optical grade silicone material is a flexible transparent material with outstanding optical properties (absorption, stability, radiation resistance, heat resistance). It is already been used in automotive headlamps as silicone lenses as they can withstand high temperatures and high UV transmission without any optical degradation. Moreover, the low absorption of optical grade silicone makes it possible to use the material as flexible radiation guide.

A known silicone radiation guiding device 10 is schematically illustrated in an elevated top view in FIG. 1, and in cross section in FIG. 2. Compact radiation sources 20, for instance LEDs, emit radiation in the centre 11 of radiation device 10. The emitted radiation is coupled in at the sides of a radiation guide 12, and the coupled-in radiation is conducted through the silicone radiation guide 12 and coupled out on the skin side 23 of the radiation device 10. Use of a reflector 24 on the top side of the silicone radiation guiding device (e.g. high reflective white silicone material or other type of reflector), preferably with an air gap 25 between the reflector 24 and the radiation guide 12, assists transferring the radiation to the end of the tapered radiation guide by means of total internal reflection.

Since the moisture permeability of 1 mm thick silicone is around 100 g/m²×24 hours (data from Dow Corning), and assuming that the maximum amount of sweat moisture on 100 cm² skin area is about 10 ml per hour, the permeability is a factor of 250 too small to carry away skin moisture through the silicone radiation guide. Consequently, the silicone radiation guide can stick to the human body with sweat in between, creating an irritating skin. There is thus room for improved radiation devices with breathable intermediate optical layers.

US2007/0239232 A1 describes a phototherapy device based on a light guide for irradiating skin. The device has a side-lit illumination configuration wherein radiation is coupled in side-ways, transported in a light guide and coupled out using outcoupling structures such as for example scatter beads. In order to enable transferring moisture vapor from the skin, vapour channels are provided in the phototherapy device.

### SUMMARY OF THE INVENTION

It is an object of the present invention to provide efficient wearable radiation devices with good user comfort. It is an advantage of embodiments according to the present invention that compact radiation devices are provided, allowing at the same time efficient outcoupling and good transfer of moisture from the skin to the environment. It is an advantage of at least some embodiments according to the present invention that wearable radiation devices are provided with reduced complexity, e.g. devices wherein less features need to be introduced during manufacturing. It is an advantage of at least some embodiments according to the present invention that efficient radiation devices are provided that at the same time can avoid or reduce skin irritation. It is an advantage of at least some embodiments of the present invention that radiation devices can be provided wherein the power density can be substantially constant over the surface. It is an advantage of embodiments of the present invention that therapy devices can be provided wherein radiation can be very efficiently used for irradiating skin.

The above objective is accomplished by a method and device according to the present invention.

In a first aspect, the present invention relates to a radiation device for providing radiation to the skin. According to embodiments of the present invention, the device comprises a radiation guide for directing radiation, the radiation guide being configured for receiving radiation from at least one radiation source in a side-lit configuration, and moisture transfer channels inside the radiation guide for enabling moisture transfer from the skin through the radiation guide to the environment. In embodiments of the present invention, the shape of the walls of the moisture transfer channels is furthermore adapted for redirecting radiation in the radiation guide towards the skin. It is an advantage of embodiments according to the present invention that efficient outcoupling as well as vapour transfer can be obtained simultaneously using the same features in the radiation device, resulting in a less complex manufacturing of the device and a sufficient to high optical efficiency. The radiation guide may be made of optical grade silicone material, e.g. polydimethylsiloxaan (PDMS), although also other optical grade flexible materials such as thermoplastic elastomer (TPE), thermoplastic polyurethane (TPU) or polyurethane could be used. According to embodiments of the present invention, the walls of the moisture transfer channels may be inclined over an angle with respect to the surface of the radiation guide facing the skin when being used, so that radiation from the at least one radiation source in side-lit configuration incident on the walls of the moisture transfer channels is redirected towards the skin. Moreover, the angle of inclination of the walls of the moisture transfer channels may be selected so that the radiation incident on the walls of the moisture transfer channels is maximally redirected for incidence in a direction perpendicular to the skin, for example by considering the radiation having the most frequently occurring angle of incidence. It is an advantage of embodiments according to the present invention that the angle of the walls of the moisture transfer channels with respect to the bottom surface (skin side) of the radiation guide can be chosen and optimized so that, for the incidence angle occurring most frequently for incidence on the walls, the radiation is either maximally transmitted through the transfer channel, or maximally redirected in a preferred direction by reflection at the channels walls, e.g. by total internal reflection or partial reflection.

According to embodiments of the present invention, the density of the moisture transfer channels inside the radiation guide may furthermore be adapted for generating a constant power density of the radiation on and across the skin. It is an advantage of embodiments according to the present invention that the power density on the skin can be tuned by modifying the density of the moisture transfer channels and by adapting the shape of the channels (e.g. the size and the angle of the channel wall with respect to the bottom surface of the radation guide system). Moreover, the size and the distribution of the moisture transfer channels in the radiation guide may be selected to optimize vapor evacuation of moisture from the skin to the environment.

In a radiation device according to the present invention, the radiation guide may have a wedged shape for allowing a homogeneous distribution of the radiation across the skin. Alternatively, other outcoupling structures also may be used for obtaining a homogeneous distribution. In one embodiment, the radiation guide may be a plan parallel shaped radiation guide. It is an advantage of some embodiments according to the present invention that efficient redirection of the incoupled radiation is achieved by means of additional features, such as for example moisture transfer channels, so that there is no longer need for wedge shaped radiation guide structures.

In a radiation device according to the present invention, the moisture transfer channels may be radial or tangential arranged in the radiation guide. The moisture transfer channels may be shaped, positioned or distributed to provide a better spread of the radiation and a more directed and efficient out coupling of the radiation through the radiation guide.

In a radiation device according to the present invention, the moisture transfer channels may extend completely through the thickness of the radiation guide. Alternatively, the moisture transfer channels may be recessed in the radiation guide, not going completely through the thickness of the radiation guide.

A radiation device according to the present invention may comprise a barrier layer positioned between the radiation guide and the skin when the device is in use, whereby the barrier layer has a higher moisture vapor transfer rate than the material of the radiation guide.

Furthermore, a radiation device according to the present invention may comprise additional channels in a bottom portion of the radiation guide facing the skin when the device is in use, whereby the additional channels are suitable for transferring moisture towards the moisture transfer channels.

In a radiation device according to the present invention, a reflector may be placed on top of the radiation guide, wherein an air gap is provided between the reflector and the radiation guide for allowing total internal reflection in the radiation guide. The reflector may be used for reflecting radiation guided through the light guide but not total internally reflected and may be used for reflecting radiation that had been reflected by the skin. The air gap between the reflector and the radiation guide may be maintained by making use of structures with a certain height that are moulded on the radiation guide. The air gap between the reflector and the radiation guide may be maintained by making use of structures with a certain height that are afterwards printed on the radiation guide. Alternatively, the air gap between the reflector and the radiation guide may be maintained by making use of structures with a certain height that are moulded on the reflector. The air gap between the reflector and the radiation guide may be maintained by making use of structures with a certain height that are afterwards printed on the reflector. The air gap between the reflector and the radiation guide may be maintained by making use of an intermediate layer between the reflector and the radiation guide. In a preferred embodiment, holes may be provided in the reflector for allowing moisture to be evacuated more easily from the moisture transfer channels. In yet another preferred embodiment, the air gap between the radiation guide and the reflector may be adapted for allowing moisture to escape via various evaporating routes through the reflector.

In one embodiment of a radiation device according to the present invention, the radiation device may be a photo therapy patch. It is an advantage of embodiments according to the present invention that even when a substantial amount of heat is generated in the radiation guide layer, for example due to a low efficiency of the radiation source and high residual heat dissipation from the radiation source, moisture transfer channels or dedicated breathing channels can be provided which improve the breathability of the radiation device.

In a second aspect, the present invention also relates to the use of a radiation device according to embodiments of the present invention for radiation treatment.

Particular and preferred aspects of the invention are set out in the accompanying independent and dependent claims. Features from the dependent claims may be combined with features of the independent claims and with features of other dependent claims as appropriate and not merely as explicitly set out in the claims. For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

### BRIEF DESCRIPTION OF THE DRAWINGS

FIG. 1 is a schematic elevated top view of a radiation device for irradiating skin as known from prior art.

FIG. 2 is a cross-sectional view of the radiation device as illustrated in FIG. 1.

FIG. 3 is a schematic elevated top view of a wedge-shaped radiation device comprising vapor channels with optical redirecting functionality according to a first embodiment of the present invention.

FIG. 4 is a schematic elevated top view of an alternative embodiment of a radiation device according to the present invention, where the radiation guide has a plan parallel shape comprising vapour channels with optical redirecting functionality.

FIG. 5 illustrates different radiation devices (a, b, c, d) comprising a radiation guide with specially shaped vapour channels according to embodiments of the present invention.

FIG. 6 is a cross-sectional view of an embodiment of the present invention with an additional channel for extra breathability.

FIG. 7 is a cross-sectional view of an alternative embodiment of the present invention, where the radiation device comprises a hole in the reflector and in the radiation guide, and where structures on the radiation guide maintain the air gap between the reflector and the radiation guide.

FIG. 8 is a cross-sectional view of an alternative embodiment of the present invention, where the radiation device comprises a hole in the reflector and in the radiation guide, and where structures on the reflector maintain the air gap between the reflector and the radiation guide.

FIG. 9 is a cross-sectional view of an alternative embodiment of the present invention, where the radiation device comprises a hole in the reflector and in the radiation guide, and where an intermediate layer maintains the air gap between the reflector and the radiation guide.

FIG. 10 is a cross-sectional view of a radiation thereby device according to an embodiment of the present invention wherein an additional barrier layer is provided for transporting moisture towards the vapor channels in the radiation guide.

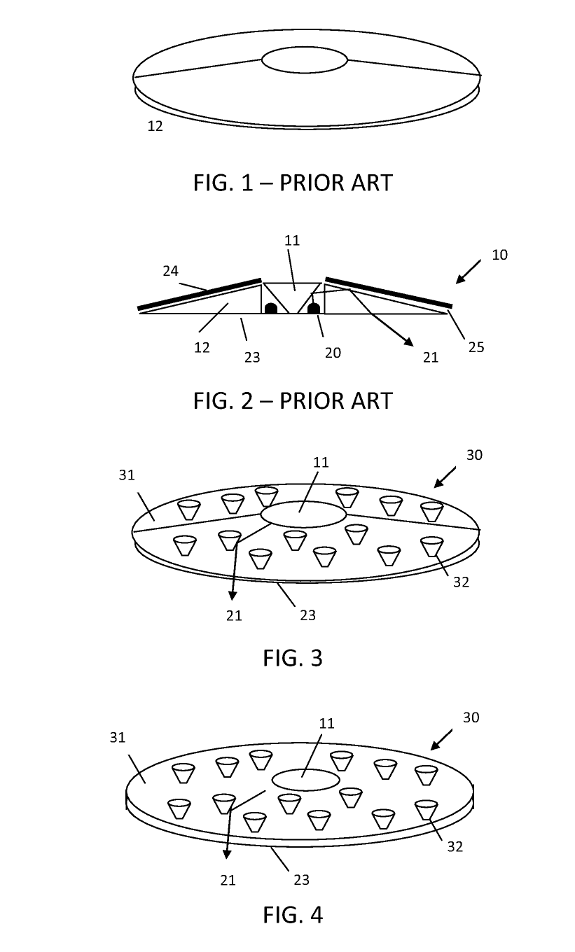

The drawings are only schematic and are non-limiting. In the drawings, the size of some of the elements may be exaggerated and not drawn to scale for illustrative purposes.

Any reference signs in the claims shall not be construed as limiting the scope. In the different drawings, the same reference signs refer to the same or analogous elements.

### DETAILED DESCRIPTION OF ILLUSTRATIVE EMBODIMENTS

Where in embodiments according to the present invention reference is made to a radiation device, reference is made to a device having provisions for radiation of the skin. Such devices may be designed as or incorporated in patches or plasters.

Where in embodiments according to the present invention reference is made to radiation, reference may be made to all types of radiation suitable for medical, wellness or cosmetic applications, such as for example infrared radiation, visible light, ultraviolet radiation, etc.

Where in embodiments according to the present invention reference is made to a wearable device, reference is made to a device that can be worn by the user during application, so that the user is free to move.

In a first aspect, the present invention describes wearable radiation device. Such devices may especially be suitable for radiation therapy for skin, such as for example in heat therapy for skin, although embodiments of the present invention are not limited thereto and are suitable for different medical applications, wellness applications, cosmetic applications, etc. The radiation devices may for example be applied to the skin of a living creature, such as for example to the skin of a human being or the skin of an animal, e.g. a horse leg. The radiation therapy system according to embodiments of the present invention comprises a radiation guide for guiding radiation towards the skin and moisture transfer channels for transferring moisture from the skin to the environment during application of the radiation device and for redirecting radiation in the radiation guide towards the skin. According to embodiments of the present invention, the radiation guide is configured for receiving radiation from at least one radiation source in a side-lit configuration. In some embodiments, a plurality of radiation sources may be used. Using a side-lit configuration, i.e. a configuration wherein radiation enters the radiation guide and is substantially spread laterally in the radiation guide before coupling out the radiation from the radiation guide, allows making the radiation device compact and flat, resulting in an increased user comfort and wearability. The concentration of multiple radiation sources on one substrate, occupying an area which is substantially smaller than the size of the radiation guide, makes the radiation device cheap and practical for production. The radiation sources may be positioned at the edge of the radiation guide, in a central recess in the radiation guide or in a plurality of recesses in the radiation guide. The at least one radiation source typically may be a light emitting device (LED), although also other radiation sources such as GLS, halogen or fluorescent sources may be used. The at least one radiation source may be part of the radiation devices according to embodiments of the present invention but does not need to be part thereof. Alternatively, the radiation devices may be adapted for receiving the at least one radiation source and to co-operate therewith. The wavelength or wavelength range of the radiation sources used may depend on the application. For example, for the treatment of wrinkles, possibly in combination with an anti-wrinkle crme, amber radiation with a wavelength around 580 nm could be used; for the disinfection of wounds, UV radiation or blue light with a wavelength around 430 nm typically could be use; for the local treatment of Acne, blue light in the wavelength range 400 nm to 440 nm could be used; for the treatment of Psoriasis or Vitiligo, advantageously UV radiation with a wavelength of or around 311 nm could be used; for skin cancer, Lupus radiation with a wavelength of around 365 nm could be used; for pain relief advantageously blue and/or infrared radiation could be used, etc.

The radiation guide may be made of any suitable material that is transparent for the radiation used and allows good contact with the skin. Advantageously, the material is soft or flexible. As indicated above, the radiation guide typically may for example be made of a silicone material, e.g. polydimethylsiloxaan (PDMS), although also other optical grade flexible materials such as thermoplastic elastomer (TPE), thermoplastic polyurethane (TPU) or polyurethane could be used. The size of the radiation guide used may depend on the application. Typically, the thickness of the radiation guide at the radiation in-coupling side may be at least 1.5 times the diameter of the radiation source (for example 4 to 6 mm) and will generally decrease towards the opposite side of the radiation guide, until a thickness of for example smaller than 1 mm, e.g. smaller than 0.1 mm is achieved. As will be illustrated and discussed in more detail with respect to particular examples below, the radiation guide may be provided with reflecting layers and/or shaped in a special manner, e.g. for further assisting in distribution of the radiation across the radiation guide.

The radiation guide according to embodiments of the present invention comprises moisture transfer channels for enabling moisture transfer from the skin through the radiation guide to the environment. Such moisture transfer channels may be provided as holes in the radiation guide. In some advantageous embodiments these holes may extend throughout the full thickness of the radiation guide (i.e. through-holes), whereas in other embodiments these holes are provides as recesses in the radiation guide (i.e. blind holes), thus providing channels for moisture transfer through at least part of the radiation guide for example for better evacuation of moisture from the skin. Moisture transfer channels implemented as blind holes may also be used in combination with the moisture permeability property of the radiation guide material to effectively transfer skin moisture to the environment. Typically, a plurality of moisture transfer channels, possibly of different types or geometries, may be provided, embodiments of the present invention being not limited by the exact number of moisture transfer channels. The size and the distribution of the moisture transfer channels may be adapted to improve the vapor evacuation of moisture. The moisture transfer channels may have for example an average diameter between 0.5 mm and a few mm, although embodiments of the present invention are not limited thereto.

According to embodiments of the present invention, the moisture transfer channels have a shape that is adapted for redirecting, at their walls, radiation present in the radiation guide towards the skin. The shape of the moisture transfer channels may be such that radiation hitting the wall of the moisture transfer channel is redirected to the bottom side of the radiation guide, in contact with or facing the skin during application. In some embodiments according to the present invention, the moisture transfer channels may have their smallest opening at the side of the radiation device that is in contact with the skin during application and their largest opening at the side of the radiation device opposite to the side of the radiation device that is in contact with the skin during application. The wall of the moisture transfer channel may typically be slanted, i.e. make an inclined angle different from 90°, with respect to the bottom surface of the radiation device. In the case of a silicone radiation guide, the radiation guide may for example be configured such that the incident radiation on the wall is inclined with an angle of about 42° or more in respect to the normal surface of the wall of the moisture transfer channel, so that total reflection takes place and the radiation is maximally redirected towards the skin. Moreover, the holes can be shaped in such way that the radiation is coupled out substantially perpendicularly to the skin, leading to a more efficient system for radiation therapy. An angle of incidence on the skin that is substantially perpendicular to the skin will reduce the amount of radiation reflected back from the skin. In a particular embodiment of the present invention, the density of the moisture transfer channels inside the radiation guide may furthermore be adapted to generate a constant power density over the skin. Radiation power density across the skin is a.o. determined by inclination angle of the moisture transfer channels and the density, i.e. number of channels per unit area, of the moisture transfer channels across the radiation guide.

By way of illustration, embodiments of the present invention not being limited thereby, further details of standard and/or optional features of a radiation device are discussed below with reference to exemplary embodiments.

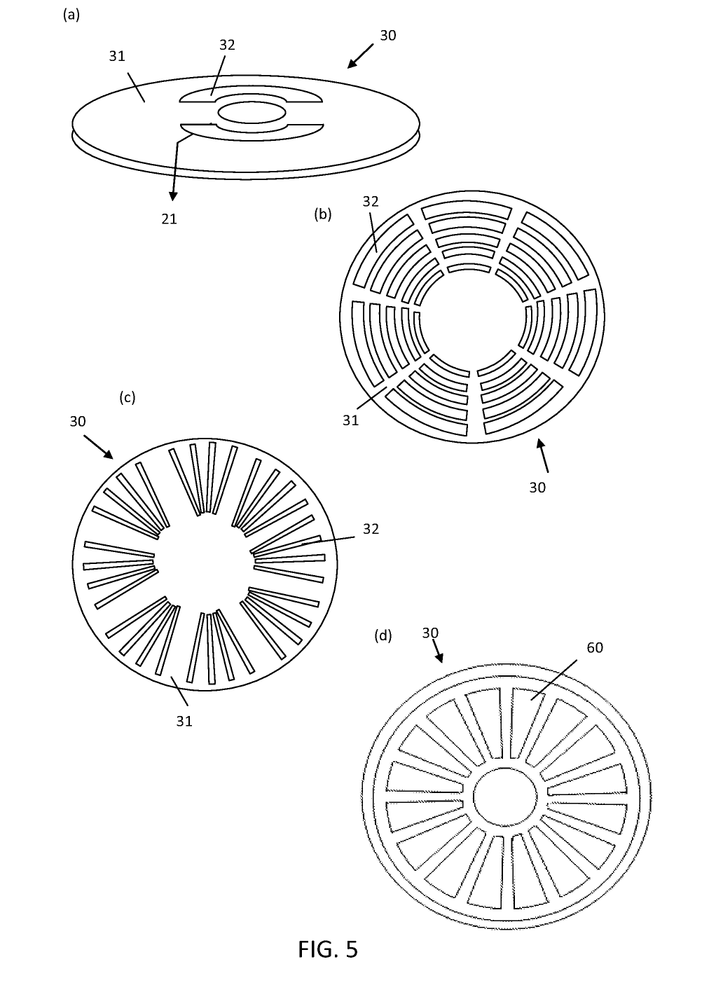

A radiation device 30 according to a first embodiment of the present invention is illustrated in FIG. 3. The radiation device 30 comprises a radiation guide 31, through which the radiation, emitted by at least one radiation source, in the present example being provided in a central recess 11 of the radiation guide system 30, is conducted towards the skin side 23 of the radiation guide device 30 (i.e. bottom side of FIG. 3). In a particular embodiment of the present invention, the radiation guide 31 may have a wedged shape for assisting in a lateral heterogeneous distribution of the radiation across the skin. In alternative embodiments, however, as illustrated in FIG. 4, the radiation guide 31 may be a plan parallel shaped radiation guide, enhancing the manufacturability of the radiation device 30. The radiation guide 31 comprises moisture transfer channels 32. The moisture transfer channels 32, introduced as holes 32 in the radiation guide of the present example, allow the moisture, produced at the skin side 23, to evaporate from the skin side 23 of the radiation device 30 and evacuated towards the environment, making the radiation device 30 a breathable construction. As discussed above, apart from their moisture transfer function, according to embodiments of the present invention, the channels 32 also have a radiation guidance function, by serving as redirecting structures for directing the radiation towards the skin. The radiation 21, which is coupled in at the center 11 of the radiation guide, is reflected by the walls of the moisture transfer channels 32, and is redirected to the bottom surface of the radiation guide, i.e. towards the side 23 where the radiation device is in contact with the skin. In the embodiments shown in FIG. 3 and FIG. 4, the moisture transfer channels 32 are holes extending throughout the radiation guide. The moisture transfer channels in the example shown have a truncated conical shape, whereby the moisture transfer channels have their smallest opening at the side where the radiation device is in contact with the skin and their largest opening at the upper side of the radiation device 31, opposite the surface that is in contact with the skin during application. The walls of the moisture transfer channels are slanted with respect to the longitudinal direction of the radiation guide (i.e. main radiation propagation direction of the radiation guide) which is substantially parallel to the bottom surface of the radiation device, resulting in the redirecting effect for the radiation.

In yet another embodiment of the present invention, some or all of the moisture transfer channels 32 inside the radiation guide 31 may be specially shaped to provide a better spreading of the radiation 21 across the radiation guide and a better efficiency of the radiation device 30 as a whole. In some examples, the moisture transfer channels are located center symmetrical, although embodiments of the present invention are not limited thereto. Some examples of different shapes of moisture transfer channels 32 are illustrated in FIG. 5. FIG. 5(a) shows a radiation device 30 with two moisture transfer channels 32, center symmetrically arranged in the radiation guide 31. The bottom and top surface of the moisture transfer channels thereby have the shape of a ring segment. The moisture transfer channels may be rectangular in cross-section. The shape of the moisture transfer channels may be selected so that radiation can go as much as possible across a moisture transfer channel without disturbance and to be optionally guided further through the radiation guide.

Efficient outcoupling of the radiation towards the skin may be obtained by making the radiation guide structure tapered (i.e. by gradually decreasing the thickness of the radiation guide layer 31 from the incoupling side towards the end side). An improved out-coupling or redirection of the radiation can furthermore be achieved by configuring the radation guide system in such way that the walls of the moisture transfer channels are slanted with respect to the out-coupling surface of the radiation guide, which is in the embodiments illustrated in the figures the bottom surface of the radiation device. In the case of a silicone radiation guide layer, the radiation device may for example be configured such that incident radiation is inclined with an angle of about 42° or more in respect to the normal surface of the wall of the moisture transfer channel, so that total reflection takes place and the radiation is maximally redirected towards the skin. FIG. 5(b) shows a radiation guide system 30 with multiple moisture transfer channels 32, center symmetrical and tangential arranged in the radiation guide 31. The top and bottom ends (openings) of the moisture transfer channels are shaped as parts of a ring. FIG. 5 (c) shows a radiation guide system 30 with multiple moisture transfer channels arranged in radial directions across the radiation guide 31. The moisture transfer channels have a top and bottom opening being rectangular in shape, and the walls of the moisture transfer channels 32 are slanted, i.e. make an inclined angle different from 90°, with respect to the radiation out-coupling surface of the radiation guide, which is in this embodiment parallel to the bottom surface of the radiation device, for providing the radiation redirecting effect.

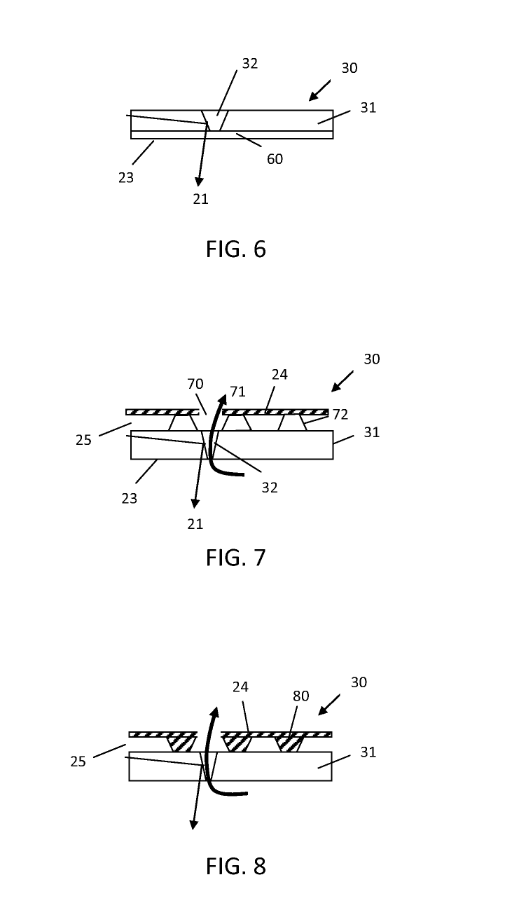

In another alternative embodiment of the present invention, the radiation device 30 is provided with additional channels 60 in the radiation guide at the side in contact with the skin. Such channels may be recesses in the bottom surface of the radiation device 30, the bottom surface is typically in contact with the skin during application. By way of illustrative embodiments, the present invention not being limited thereto, a radiation device 30 with additional channels 60 in the radiation guide is shown in cross-section in FIG. 6. The additional channels 60 typically do not extend through the full thickness of the radiation guide 31. The number and size of channels typically may be selected such that still a large part, e.g. at least 50%, of the radiation guide 31 is in direct contact with the skin. The moisture channels may extend to the full thickness, or to part of it. Part of the channels may be oriented so that airflow and moisture transport to the side of the radiation guide is promoted. The additional channels may be configured such that a spokes-structure configuration is obtained. An example thereof is shown in FIG. 5(d). The additional channels 60 may be in direct contact with the moisture transfer channels such that moisture captured in the additional channels can be transferred from the additional channels 60 to the moisture transfer channels 32 out of the radiation device 30. The additional channels 60 may be placed in a plane substantially perpendicular to the direction of the moisture transfer channels 32. They may be placed at any suitable position and in any suitable direction in the bottom part of the radiation guide being in contact with the skin when the radiation device is in use, such as for example in radial or tangential direction. The presence of additional channels allows for extra breathability of the radiation therapy device 30. In an exemplary embodiment of a radiation device 30, a configuration of additional channels 60 as illustrated in FIG. 5(d) may be combined with moisture transfer channels 32 configured according to FIG. 5(b).

A radiation device according to embodiments of the present invention may furthermore comprise a reflecting layer or reflector 24, placed on top of the radiation guide 31. The reflecting layer or reflector 24 is adapted for redirecting radiation towards the skin. Moreover, the reflecting layer reflects radiation 21 that was reflected from the skin, back to the skin. The reflecting layer or reflector 24 may be made of any suitable reflective material, such as for example a metallic reflector or a reflecting stack of dielectric layers. The reflecting layer 24 may for example also be made from a high reflective white silicone material. An air gap 25 may be provided between the reflecting layer or reflector 24 and the radiation guide 31, so that good conditions are created for occurrence of total internal reflection, while, in case the radiation 21 is not reflected but coupled out, this radiation is redirected in the radiation guide again by the reflecting layer.

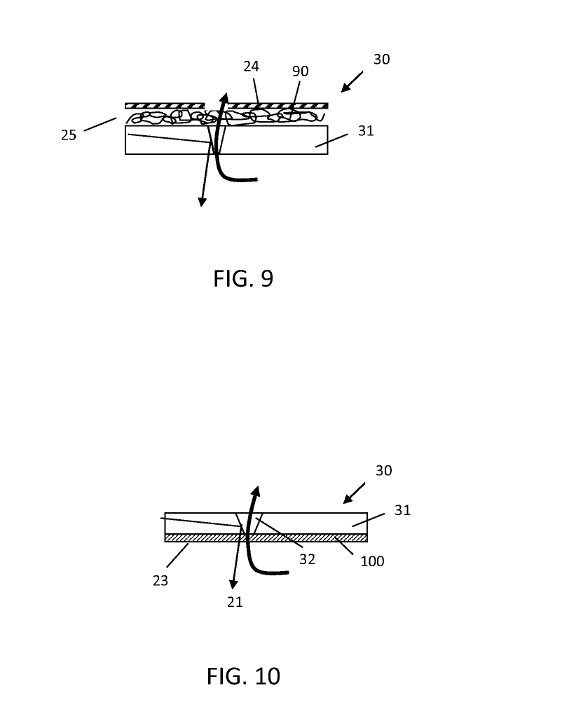

An example of a radiation device 30 comprising a reflecting layer or reflector 24, a radiation guide 31, and a moisture transfer channel 32 in the radiation guide 31, is illustrated in cross-section in FIG. 7. In some embodiments, the moisture transfer channels 32 do not only extend through or are present in the radiation guide 31, but also extend through the reflecting layer or reflector 24. More particularly, an additional hole 70, located above the moisture transfer channel 32 in the radiation guide 31 may be foreseen in the reflecting layer or reflector 24 so that moisture present in the moisture transfer channel 32 can escape through the additional hole 70. The additional holes in the reflector and the moisture transfer channels 32 in the radiation guide 31 thus make the system breathable for the skin, by providing an evaporating escape route 71 for moisture coming from the skin side 23 of the radiation device 30. In the embodiment illustrated in FIG. 7, the air gap 25 between the reflecting layer or reflector 24 and the radiation guide 31 is maintained by a spacer 72. Such a spacer may for example be comprised of structures molded on the radiation guide, structures provided on the reflecting layer or reflector, an intermediate layer, etc. In FIG. 7 the spacer provided is a spacer making use of structures 72 with a certain height that are moulded on the radiation guide 31. Alternatively, the structures 72 may afterwards be printed onto the radiation guide 31 by means of screen-printing or hot embossing. The height and the distance of the structures 72 may be chosen in such a way, that a predefined air gap 25 is maintained. The structures 72 may be made from a transparent or a white high reflective material. FIG. 8 illustrates the case that the spacer is provided by structures provided on the reflector. More particularly, the air gap 25 between the reflector 24 and the radiation guide 31 may be maintained by making use of structures 80 that are moulded on the reflector 24. Alternatively, the structures 80 may afterwards be printed onto the reflector 24 by means of screen-printing or hot embossing. The structures 80 may be of the same material as the reflector 24. In one embodiment, the structures 80 may be made from white high reflective material. FIG. 9 illustrates the case wherein the spacer is an intermediate layer 90 placed between the radiation guide 31 and the reflector 24 to maintain the air gap 25. The intermediate layer 90 may be a high transparent textile layer. In yet another embodiment, the intermediate layer 90 may be a silica layer.

In another alternative embodiment of the present invention, the air gap 25 between the radiation guide 31 and the reflector 24 may be made large enough, allowing the moisture to escape horizontally towards the sides and follow various evaporating routes through the reflector 24. Layers on top of the reflector 24 (heat spreading materials, cover layers, etc.) than advantageously also have open structures or holes, allowing the moisture to evaporate to the environment and providing breathing channels for the skin.

In yet another alternative embodiment of the present invention, the radiation device 30 may comprise a barrier layer, e.g. a porous membrane 100, arranged to be in between the skin 23 and the radiation guide 31, when the radiation device is in use. This is illustrated in cross-section in FIG. 10. The barrier layer 100 may be provided as a layer that allows a fair amount of moisture vapor coming from the skin side 23 of the radiation device 30 to travel laterally to reach at least one of the holes 32 of the radiation guide layer 31. If the moisture transfer rate in the barrier layer is higher than the moisture transfer rate in the radiation guide material and moisture transfer channels, this results in an enhancement of the moisture evacuation efficiency of the radiation device 30. The barrier layer 100 could either be permanent with the radiation guide layer 31, or removable, and optionally replaceable. The barrier layer 100 could also be a multi-layer structure, having for example layers having an increasing moisture vapor transfer rate from the skin towards the radiation guide. When the barrier layer 100 is made from a breathable material, channel 32 may also serve as an additional air channel for ventilation of the skin.

In one aspect, the present invention relates to the use of a radiation device according to the first aspect, e.g. as described in any of the above embodiments, for irradiating skin of a living creature. The irradiation may be performed for a plurality of applications as described above, e.g. for cosmetic, medical or wellness applications. The use of a radiation device as described herein may assist in providing an efficient irradiation of the skin, while avoiding irritation of the skin by transferring moisture from the surface of the skin, through the radiation device, towards the environment.

While the invention has been illustrated and described in detail in the drawings and foregoing description, such illustration and description are to be considered illustrative or exemplary and not restrictive. The invention is not limited to the disclosed embodiments.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

The foregoing description details certain embodiments of the invention. It will be appreciated, however, that no matter how detailed the foregoing appears in text, the invention may be practiced in many ways. It should be noted that the use of particular terminology when describing certain features or aspects of the invention should not be taken to imply that the terminology is being re-defined herein to be restricted to include any specific characteristics of the features or aspects of the invention with which that terminology is associated.

1. A radiation device for providing radiation to the skin, the device comprising:
a radiation guide for directing radiation, the radiation guide being configured for receiving radiation from at least one radiation source in a side-lit configuration wherein radiation enters the radiation guide at a side thereof and is spread in the radiation guide substantially laterally, the radiation guide comprising:
at least one moisture transfer channel for enabling moisture transfer from the skin through the radiation guide to the environment,
wherein a shape of a wall of the at least one moisture transfer channel is adapted for redirecting radiation received in the radiation guide towards the skin.

2. A radiation device according to claim 1, wherein the wall of the at least one moisture transfer channel is inclined over an angle of inclination with respect to an out-coupling surface of the radiation guide facing the skin, when being used, so that radiation from the at least one radiation source which is incident on the wall of the at least one moisture transfer channel is redirected towards the skin.

3. A radiation device according to claim 2, wherein the angle of inclination of the wall of the at least one moisture transfer channel is selected so that the radiation incident on the wall of the at least one moisture transfer channel is maximally redirected for incidence in a direction perpendicular to the skin.

4. A radiation device according to claim 1, the radiation guide comprising multiple moisture transfer channels wherein the density of the moisture transfer channels is adapted for generating a constant power density of the radiation across the skin.

5. A radiation device according to claim 1, wherein the radiation guide is wedge shaped for allowing a homogeneous distribution of the radiation across the skin.

6. A radiation device according to claim 1, wherein the radiation guide is a plan-parallel shaped.

7. A radiation device according to claim 1, wherein the at least one moisture transfer channel is radial or tangential arranged in the radiation guide (31).

8. A radiation device according to claim 1, wherein the at least one moisture transfer channel is extending completely through the thickness of the radiation guide.

9. A radiation device according to claim 1, wherein the at least one moisture transfer channel is recessed in the radiation guide.

10. A radiation device according to claim 1, further comprising a barrier layer arranged to be in between the radiation guide and the skin, when the device is in use, and wherein the barrier layer has a higher moisture vapor transfer rate than a material of the radiation guide.

11. A radiation device according to claim 1, wherein an additional channel is provided in a bottom portion of the radiation guide facing the skin, when the device is in use, the additional channel being suitable for transferring moisture towards the moisture transfer channels.

12. A radiation device according to claim 1, furthermore comprising a reflector positioned adjacent a top surface of the radiation guide, the top surface being opposite a surface of the radiation guide facing the skin, when the devices is in use, and wherein an air gap is provided between the reflector and the top surface of the radiation guide for allowing total internal reflection in the radiation guide.

13. A radiation device according to claim 1, wherein holes are present in the reflector for allowing moisture from the moisture transfer channels to be evacuated through the holes.

14. A radiation device according to claim 12, wherein the air gap between the radiation guide and the reflector is adapted for allowing moisture to escape via various evaporating routes through the reflector.

15. Use of a radiation device according to claim 12 for radiation treatment.
